(19) United States
(12) Patent Application Publication
(10) Pub. No.: US 2003/0196219 A1
(43) Pub. Date: Oct. 16, 2003

(54) Novel Method for Agrobacterium Preparation for Plant Transformation

(75) Inventor: Wanggen Zhang, Wildwood, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(73) Assignee: MONSANTO TECHNOLOGY LLC

(21) Appl. No.: 10/249,517

(22) Filed: Apr. 16, 2003

Related U.S. Application Data
(60) Provisional application No. 60/319,192, filed on Apr. 16, 2002.

(51) Int. Cl. A01H 1/00; C12N 15/82; C12N 1/20

(52) U.S. Cl. 800/294; 435/252.2

(57) ABSTRACT

The present invention relates to a novel method of preparing Agrobacterium for plant transformation. In particular, the invention relates to storing the Agrobacterium in the cold for some period of time. Surprisingly, this increases transformation efficiency.

This application claims priority to U.S. Provisional Application No. 60/319,192, filed Apr. 16, 2002, incorporated by reference herein in its entirety.

### BACKGROUND OF THE INVENTION

The present invention relates to the field of plant biotechnology. More specifically, it concerns methods of improving the process of incorporating genetic components into a plant via an Agrobacterium—mediated process.

The ability to transfer genes from a wide range of organisms to crop plants by recombinant DNA technology has become widespread in recent years. This advance has provided enormous opportunities to improve plant resistance to pests, disease and herbicides, and to modify biosynthetic processes to change the quality of plant products. Highly efficient methods for transformation of these crop plants continues to be a goal as there is a need for high capacity production of economically important plants.

Agrobacterium—mediated transformation is one method for transforming such crop plants and has more recently become more adaptable for use in monocotyledonous plants. Several Agrobacterium species mediate the transfer of a specific DNA known as “T-DNA”, that can be genetically engineered to carry a desired piece of DNA into the selected plant species. The major events marking the process of T-DNA mediated pathogenesis and ultimate transformation are induction of virulence genes, processing and transfer of the T-DNA to the plant's genome.

Typically, Agrobacterium—mediated genetic transformation of plants involves several steps. The first step, in which the Agrobacterium and plant cells are first brought into contact with each other, is generally called “inoculation.” Following the inoculation step, the Agrobacterium and plant cells/tissues are usually grown together for a period of several hours to several days or more under conditions suitable for growth and T-DNA transfer. This step is termed “co-culture”. Following co-culture and T-DNA delivery, the plant cells are often treated with bactericidal or bacteriostatic agents to kill or suppress the Agrobacterium. If this is done in the absence of any selective agents to promote preferential growth of transgenic versus non-transgenic plant cells, then this is typically referred to as the “delay” step. If done in the presence of selective pressure favoring transgenic plant cells, then it is referred to as a “selection” step. When a “delay” is used, it is usually followed by one or more “selection” steps. Both the “delay” and “selection” steps typically include bactericidal or bacteriostatic agents to kill or suppress any remaining Agrobacterium cells because the growth of Agrobacterium cells is undesirable after the infection (inoculation and co-culture) process.

Prior to the inoculation step, the Agrobacterium is prepared for use. Agrobacterium is generally stored in a glycerol stock solution in the freezer. Traditionally, this material is then grown in a liquid media until it reaches a logarithmic growth phase and then is used immediately. The process of going from the stock solution to the liquid culture takes 3 to 4 days, thus limiting the flexibility of planning transformation experiments.

The present invention provides novel Agrobacterium preparation conditions that result in increased transformation efficiencies and improvement in material handling in plant transformation processes. Use of the method of the present invention results in the desired transgenic events being obtained while reducing the effort required for the transformation of such plants. The present invention thus provides a novel improvement compared to existing Agrobacterium—mediated transformation methods.

### SUMMARY OF INVENTION

The present invention provides novel conditions during the preparation of the Agrobacterium prior to inoculation with the plants cells or tissue to be transformed. The method can be used for introducing selected nucleic acids into transformable cells or tissues to provide or create a desirable trait in a plant regenerated from such cells or tissue. The present invention also provides transgenic plants made by the method of the invention, in particular, monocotyledonous plants, e.g., corn, wheat and rice. In other aspects, the invention relates to the production of stably transformed fertile plants, gametes, and offspring from these plants.

In one embodiment of the present invention, a method of transforming a plant cell or tissue by an Agrobacterium—mediated process is provided wherein the Agrobacterium is stored in a cold environment to chill the Agrobacterium solution immediately prior to its use to inoculate plant cells or tissue.

Still another embodiment of the present invention relates to transformed plants produced by the method of the present invention.

Yet another embodiment of the present invention relates to any seeds, or progeny of the transformed plants produced by the method of the present invention.

Further objects, advantages and aspects of the present invention will become apparent from the accompanying figures and description of the invention.

### BRIEF DESCRIPTION OF DRAWINGS

FIG. 1 is a plasmid map of pMON42410.

FIG. 2 is a plasmid map of pMON42411.

FIG. 3 is a plasmid map of pMON42073.

FIG. 4 is a plasmid map of pMON18365.

### DETAILED DESCRIPTION

The present invention provides a method of Agrobacterium preparation in which the liquid Agrobacterium culture is stored in a cold environment for a period of time of at least about 12 hours or from about 12 hours to about 30 days or from about 12 hours to about 21 days or from about 2 to 9 days or from about 4 to 7 days immediately prior to its use as the vehicle for DNA transfer to a plant cell or tissue. A cold environment is used herein to mean a temperature from about 0° C. to about 12° C. or from about 1° C. to about 6° C. Typically, storage is performed in a refrigerator at about 4° C. It has been discovered that storage of the Agrobacterium in this manner increases the transformation efficiency of the plant tissue or cell being transformed and provides the technicians performing the transformation experiments more flexibility in conducting the experiments. The present invention is particularly useful for monocots, e.g., corn, wheat, and rice. The present invention provides a transgenic plant and a method for transformation of plant cells or tissues and recovery of the transformed cells or tissues into a differentiated transformed plant.

Those of skill in the art are aware of the typical steps in the plant transformation process. The Agrobacterium can be prepared either by inoculating a liquid such as Luria Burtani (LB) media directly from a glycerol stock or streaking the Agrobacterium onto a solidified media from a glycerol stock, allowing the bacteria to grow under the appropriate selective conditions, generally from about 26° C.-30° C., usually about 28° C., and taking a single colony from the plate and inoculating a liquid culture medium containing the selective agents. Alternatively a loopful or slurry of Agrobacterium can be taken from the plate and resuspended in liquid and used for inoculation. Those of skill in the art are familiar with procedures for growth and suitable culture conditions for Agrobacterium as well as subsequent inoculation procedures. The inoculation density of the Agrobacterium culture used for inoculation of the plant cells or plant tissue and the ratio of Agrobacterium cells to explant can vary from one system to the next, and therefore optimization of these parameters for any transformation method is expected. Typically, the inoculation density is between about 0.1 and about 2.0 OD at 660 nm. Agrobacterium inoculation culture is a solution, suspension or aggregation that by definition contains Agrobacterium suitable for transformation of plants.

Typically, an Agrobacterium culture is inoculated from a streaked plate or glycerol stock and is grown overnight, and the bacterial cells are washed and resuspended in a culture medium suitable for inoculation of the explant. Suitable inoculation media for the present invention include, but are not limited to, ½ MS PL or ½ MS VI (Table 1). Typically, the Agrobacterium culture is used immediately upon resuspension and then discarded at the end of the day. In the present invention, it is shown that the Agrobacterium culture can be stored in a cold environment from about 12 hours to about 30 days and retain the ability to successfully inoculate plant explants and transfer selected DNA to the explant. Surprisingly, the transformation efficiencies increase when the Agrobacterium solution is stored in a cold environment from about 3 to about 14 days, with from about 4 to about 7 days being the most efficacious. Storage is preferably done in the refrigerator at about 4° C. but temperatures can range from about 0° C. to about 12° C. and remain efficacious.

The osmotic pressure of the media could impact the survival or activity of the Agrobacterium for any length of time. Experiments wherein the Agrobacterium was suspended in a media containing 6.85% sucrose and 3.6% glucose were beneficial. A range of osmotic pressures were tested with different osmoticums to determine the survival of the Agrobacterium over the time period desired (see Example 6). The osmotic pressure does not appear to affect Agrobacterium survival. Osmoticums that may be used include, but are not limited to, sucrose, glucose, polyethylene glycol, sugars, salts, polymers, or combinations thereof. In the examples herein, the Agrobacterium was stored directly in the inoculation media. It is an aspect of this invention that the Agrobacterium could be stored with inoculation media with a higher osmotic pressure than inoculation media suitable for a particular crop and then diluted to the proper osmotic pressure immediately before use. Suitable osmotic pressure would be one at which the bacteria are viable.

The present invention encompasses the use of bacterial strains to introduce one or more genetic components into plants. Those of skill in the art would recognize the utility of Agrobacterium—mediated transformation methods. A number of wild-type and disarmed strains of Agrobacterium tumefaciens and Agrobacterium rhizogenes harboring Ti or Ri plasmids can be used for gene transfer into plants. Preferably, the Agrobacterium hosts contain disarmed Ti and Ri plasmids that do not contain the oncogenes that cause tumorigenesis or rhizogenesis, respectively, which are used as the vectors and contain the genes of interest that are subsequently introduced into plants. Preferred strains would include, but are not limited to, Agrobacterium tumefaciens strain C58, a nopaline-type strain that is used to mediate the transfer of DNA into a plant cell, octopine-type strains such as LBA4404 or succinamopine-type strains, e.g., EHA101 or EHA105. The use of these strains for plant transformation has been reported and the methods are familiar to those of skill in the art.

The present invention can be used with any transformable cell or tissue. By transformable as used herein is meant a cell or tissue that is capable of further propagation to give rise to a plant. Those of skill in the art recognize that a number of plant cells or tissues are transformable in which after insertion of exogenous DNA and appropriate culture conditions the plant cells or tissues can form into a differentiated plant. Tissue suitable for these purposes can include but is not limited to immature embryos, scutellar tissue, suspension cell cultures, immature inflorescence, shoot meristem, nodal explants, callus tissue, hypocotyl tissue, cotyledons, roots, and leaves.

Any suitable plant culture medium for transformation and regeneration can be used. Examples of suitable media would include but are not limited to MS-based media (Mursahige and Skoog, Physiol. Plant, 15:473-497, 1962) or N6-based media (Chu et al., Scientia Sinica 18:659, 1975) supplemented with additional plant growth regulators including, but not limited to, auxins such as picloram (4-amino-3,5,6-trichloropicolinic acid), 2,4-D (2,4-dichlorophenoxyacetic acid) and dicamba (3,6-dichloroanisic acid), cytokinins such as BAP (6-benzylaminopurine ) and kinetin, and gibberellins. Other media additives can include but are not limited to amino acids, macroelements, iron, microelements, vitamins and organics, carbohydrates, undefined media components such as casein hydrolysates, an appropriate gelling agent such as a form of agar, such as a low melting point agarose or Gelrite if desired. Those of skill in the art are familiar with the variety of tissue culture media, which when supplemented appropriately, support plant tissue growth and development and are suitable for plant transformation and regeneration. These tissue culture media can either be purchased as a commercial preparation, or custom prepared and modified. Examples of such media would include but are not limited to Murashige and Skoog (Mursahige and Skoog, Physiol. Plant, 15:473-497, 1962), N6 (Chu et al., Scientia Sinica 18:659, 1975), Linsmaier and Skoog (Linsmaier and Skoog, Physio. Plant., 18: 100, 1965), Uchimiya and Murashige (Uchimiya and Murashige, Plant Physiol. 15:473, 1962), Gamborg's B5 media (Gamborg et al., Exp. Cell Res., 50:151, 1968), D medium (Duncan et al., Planta, 165:322-332, 1985), McCown's Woody plant media (McCown and Lloyd, HortScience 16:453, 1981), Nitsch and Nitsch (Nitsch and Nitsch, Science 163:85-87, 1969), and Schenk and Hildebrandt (Schenk and Hildebrandt, Can. J. Bot. 50:199-204,1972) or derivations of these media supplemented accordingly. Those of skill in the art are aware that media and media supplements such as nutrients and growth regulators for use in transformation and regeneration and other culture conditions such as light intensity during incubation, pH, and incubation temperatures that can be optimized for the particular variety of interest.

Once the transformable plant tissue is isolated, the next step of the method is introducing the genetic components into the plant tissue. This process is also referred to herein as “transformation.” The plant cells are transformed and each independently transformed plant cell is selected. The independent transformants are referred to as transgenic events. A number of methods have been reported and can be used to insert genetic components into transformable plant tissue. Those of skill in the art are aware of the typical steps in the plant transformation process. The Agrobacterium can be prepared as described above and stored in a cold environment as desired.

The first stage of the transformation process is the inoculation. In this stage the explants and Agrobacterium cell suspensions are mixed together. The mixture of Agrobacterium and explant(s) can also occur prior to or after a wounding step. By wounding as used herein is meant any method to disrupt the plant cells thereby allowing the Agrobacterium to interact with the plant cells. Those of skill in the art are aware of the numerous methods for wounding. These methods would include but are not limited to particle bombardment of plant tissues, sonicating, shearing, piercing, poking, cutting, or tearing plant tissues with a scalpel, needle or other device. The duration and condition of the inoculation and Agrobacterium cell density will vary depending on the plant transformation system. The inoculation is generally performed at a temperature of about 15° C.-30° C., or about 23° C.-28° C. from less than one minute to about 3 hours. The inoculation can also be done using a vacuum infiltration system.

After inoculation any excess Agrobacterium suspension can be removed and the Agrobacterium and target plant material are co-cultured. The co-culture refers to the time post-inoculation and prior to transfer to a delay or selection medium. Any number of plant tissue culture media can be used for the co-culture step. For the present invention a reduced salt media such as ½ MS-based co-culture media (Table 1) is used, and the media lacks complex media additives including but not limited to undefined additives such as casein hydrolysate, and B5 vitamins and organic additives. Plant tissues after inoculation with Agrobacterium can be cultured in a liquid media. Alternatively, plant tissues after inoculation with Agrobacterium are cultured on a semi-solid culture medium solidified with a gelling agent such as agarose, generally a low EEO agarose. The co-culture duration is from about one hour to 72 hours, or less than 36 hours, or about 6 hours to 35 hours. The co-culture is typically performed for about one to three days or for less than 24 hours at a temperature of about 18° C.-30° C., or about 23° C.-25° C. The co-culture can be performed in the light or in light-limiting conditions. Lighting conditions can be optimized for each plant system as is known to those of skill in the art.

After co-culture with Agrobacterium, the explants can be placed directly onto selective media. Explants can be sub-cultured onto selective media in successive steps or stages. For example, the first selective media could contain a low amount of selective agent, and the next sub-culture could contain a higher concentration of selective agent or vice versa. The explants could also be placed directly on a fixed concentration of selective agent. Alternatively, after co-culture with Agrobacterium, the explants could be placed on media without the selective agent. Those of skill in the art are aware of the numerous modifications in selective regimes, media, and growth conditions that can be varied depending on the plant system and the selective agent. Typical selective agents include but are not limited to antibiotics such as geneticin (G418), kanamycin, paromomycin or other chemicals such as glyphosate. Additional appropriate media components can be added to the selection or delay medium to inhibit Agrobacterium growth. Such media components can include, but are not limited to, antibiotics such as carbenicillin or cefotaxime. The cultures are subsequently transferred to a media suitable for the recovery of transformed plantlets. Those of skill in the art are aware of the number of methods to recover transformed plants. A variety of media and transfer requirements can be implemented and optimized for each plant system for plant transformation and recovery of transgenic plants. Consequently, such media and culture conditions disclosed in the present invention can be modified or substituted with nutritionally equivalent components, or similar processes for selection and recovery of transgenic events, and still fall within the scope of the present invention.

The transformants produced are subsequently analyzed to determine the presence or absence of a particular nucleic acid of interest contained on the transformation vector. Molecular analyses can include but is not limited to Southern blots (Southern, J. Mol. Biol., 98:503-517, 1975), or PCR (polymerase chain reaction) analyses, immunodiagnostic approaches, and field evaluations. These and other well known methods can be performed to confirm the stability of the transformed plants produced by the methods disclosed. These methods are well known to those of skill in the art and have been reported (See for example, Sambrook et al., Molecular Cloning, A Laboratory Manual, 1989).

To initiate a transformation process in accordance with the present invention, it is first necessary to select genetic components to be inserted into the plant cells or tissues. Genetic components can include any nucleic acid that is introduced into a plant cell or tissue using the method according to the invention. Genetic components can include non-plant DNA, plant DNA or synthetic DNA.

In a preferred embodiment, the genetic components are incorporated into a DNA composition such as a recombinant, double-stranded plasmid or vector molecule comprising at least one or more of following types of genetic components: (a) a promoter that functions in plant cells to cause the production of an RNA sequence, (b) a structural DNA sequence that causes the production of an RNA sequence that encodes a product of agronomic utility, and (c) a 3′ non-translated DNA sequence that functions in plant cells to cause the addition of polyadenylated nucleotides to the 3′ end of the RNA sequence.

The vector may contain a number of genetic components to facilitate transformation of the plant cell or tissue and regulate expression of the desired gene(s). In one preferred embodiment, the genetic components are oriented so as to express a mRNA, that in one embodiment can be translated into a protein. The expression of a plant structural coding sequence (a gene, cDNA, synthetic DNA, or other DNA) that exists in double-stranded form involves transcription of messenger RNA (mRNA) from one strand of the DNA by RNA polymerase enzyme and subsequent processing of the mRNA primary transcript inside the nucleus. This processing involves a 3′ non-translated region that adds polyadenylated nucleotides to the 3′ ends of the mRNA.

Means for preparing plasmids or vectors containing the desired genetic components are well known in the art. Vectors typically consist of a number of genetic components, including but not limited to regulatory elements such as promoters, leaders, introns, and terminator sequences. Regulatory elements are also referred to as cis- or trans-regulatory elements, depending on the proximity of the element to the sequences or gene(s) they control.

Transcription of DNA into mRNA is regulated by a region of DNA usually referred to as the “promoter”. The promoter region contains a sequence of bases that signals RNA polymerase to associate with the DNA and to initiate the transcription into mRNA using one of the DNA strands as a template to make a corresponding complementary strand of RNA.

A number of promoters that are active in plant cells have been described in the literature. Such promoters would include but are not limited to the nopaline synthase (NOS) and octopine synthase (OCS) promoters that are carried on tumor-inducing plasmids of Agrobacterium tumefaciens, the caulimovirus promoters such as the cauliflower mosaic virus (CaMV) 19S and 35S promoters and the figwort mosaic virus (FMV) 35S promoter, the enhanced CaMV35S promoter (e35S), the light-inducible promoter from the small subunit of ribulose bisphosphate carboxylase (ssRUBISCO, a very abundant plant polypeptide). All of these promoters have been used to create various types of DNA constructs that have been expressed in plants.

Promoter hybrids can also be constructed to enhance transcriptional activity, or to combine desired transcriptional activity, inducibility and tissue specificity or developmental specificity. Promoters that function in plants include but are not limited to promoters that are inducible, viral, synthetic, constitutive, and temporally regulated, spatially regulated, and spatio-temporally regulated. Other promoters that are tissue-enhanced, tissue-specific, or developmentally regulated are also known in the art and envisioned to have utility in the practice of this invention. As described below, it is preferred that the particular promoter selected should be capable of causing sufficient expression to result in the production of an effective amount of the gene product of interest.

The promoters used in the DNA constructs (i.e., chimeric/recombinant plant genes) of the present invention may be modified, if desired, to affect their control characteristics. Promoters can be derived by means of ligation with operator regions, random or controlled mutagenesis, etc. Furthermore, the promoters may be altered to contain multiple “enhancer sequences” to assist in elevating gene expression. Examples of such enhancer sequences have been reported by Kay et al. (Science, 236:1299, 1987).

The mRNA produced by a DNA construct of the present invention may also contain a 5′ non-translated leader sequence. This sequence can be derived from the promoter selected to express the gene and can be specifically modified so as to increase translation of the mRNA. The 5′ non-translated regions can also be obtained from viral RNAs, from suitable eukaryotic genes, or from a synthetic gene sequence. Such “enhancer” sequences may be desirable to increase or alter the translational efficiency of the resultant mRNA. The present invention is not limited to constructs wherein the non-translated region is derived from the 5′ non-translated sequence that accompanies the promoter sequence. Rather, the non-translated leader sequence can be derived from unrelated promoters or genes (see, for example U.S. Pat. No. 5,362,865). Other genetic components that serve to enhance expression or affect transcription or translational of a gene are also envisioned as genetic components. The 3′ non-translated region of the chimeric constructs should contain a transcriptional terminator, or an element having equivalent function, and a polyadenylation signal that functions in plants to cause the addition of polyadenylated nucleotides to the 3′ end of the RNA. Examples of suitable 3′ regions are (1) the 3′ transcribed, non-translated regions containing the polyadenylation signal of Agrobacterium tumor-inducing (Ti) plasmid genes, such as the nopaline synthase (NOS) gene, and (2) plant genes such as the soybean storage protein genes and the small subunit of the ribulose-1,5-bisphosphate carboxylase (ssRUBISCO) gene. An example of a preferred 3′ region is that from the ssRUBISCO E9 gene from pea.

Typically, DNA sequences located a few hundred base pairs downstream of the polyadenylation site serve to terminate transcription. The DNA sequences are referred to herein as transcription-termination regions. The regions are required for efficient polyadenylation of transcribed messenger RNA (mRNA) and are known as 3′ non-translated regions. RNA polymerase transcribes a coding DNA sequence through a site where polyadenylation occurs.

In one preferred embodiment, the vector contains a selectable, screenable, or scoreable marker gene. These genetic components are also referred to herein as functional genetic components, as they produce a product that serves a function in the identification of a transformed plant, or a product of agronomic utility. The DNA that serves as a selection device functions in a regenerable plant tissue to produce a compound that would confer upon the plant tissue resistance to an otherwise toxic compound. Genes of interest for use as a selectable, screenable, or scorable marker would include but are not limited to GUS; green fluorescent protein (GFP); luciferase (LUX); antibiotic resistance genes, such as those resistant to kanamycin (nptII), hygromycin B (aph IV) and gentamycin (aac3 and aac C4); or herbicide tolerance genes (e.g., EPSPS genes capable of conferring tolerance to the chemical phosphonomethyl glycine).

A number of selectable marker genes are known in the art and can be used in the present invention. Particularly preferred selectable marker genes for use in the present invention would include genes that confer resistance to compounds such as antibiotics like kanamycin, and herbicides like glyphosate. Other selection devices can also be implemented including but not limited to tolerance to phosphinothricin, bialaphos, and positive selection mechanisms and would still fall within the scope of the present invention.

The present invention can be used with any suitable plant transformation plasmid or vector containing a selectable or screenable marker and associated regulatory elements as described, along with one or more nucleic acids expressed in a manner sufficient to confer a particular desirable trait. Examples of suitable structural genes of agronomic interest envisioned by the present invention would include but are not limited to genes for insect or pest tolerance, herbicide tolerance, genes for quality improvements such as yield, nutritional enhancements, environmental or stress tolerances, or any desirable changes in plant physiology, growth, development, morphology or plant product(s).

Alternatively, the DNA coding sequences can effect these phenotypes by encoding a non-translatable RNA molecule that causes the targeted inhibition of expression of an endogenous gene, for example via antisense- or cosuppression-mediated mechanisms (see, for example, Bird et al., Biotech Gen. Engin. Rev., 9:207-227, 1991). The RNA could also be a catalytic RNA molecule (i.e., a ribozyme) engineered to cleave a desired endogenous mRNA product. Thus, any gene that produces a protein or mRNA that expresses a phenotype or morphology change of interest is useful for the practice of the present invention.

Exemplary nucleic acids that may be introduced by the methods encompassed by the present invention include, for example, DNA sequences or genes from another species, or even genes or sequences that originate with or are present in the same species, but are incorporated into recipient cells by genetic engineering methods rather than classical reproduction or breeding techniques. However, the term exogenous is also intended to refer to genes that are not normally present in the cell being transformed, or perhaps simply not present in the form, structure, etc., as found in the transforming DNA segment or gene, or genes that are normally present yet that one desires, eg., to have over-expressed. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the plant cell, DNA from another plant, DNA from a different organism, or a DNA generated externally, such as a DNA sequence containing an antisense message of a gene, or a DNA sequence encoding a synthetic or modified version of a gene.

In light of this disclosure, numerous other possible selectable and/or screenable marker genes, regulatory elements, and other sequences of interest will be apparent to those of skill in the art. Therefore, the foregoing discussion is intended to be exemplary rather than exhaustive.

After the construction of the plant transformation vector or construct, said nucleic acid molecule, prepared as a DNA composition in vitro, is introduced into a suitable host such as E coli and mated into another suitable host such as Agrobacterium, or directly transformed into competent Agrobacterium. These techniques are well-known to those of skill in the art and have been described for a number of plant systems including soybean, cotton, and wheat (see, for example U.S. Pat. Nos. 5,569,834, 5,159,135, and WO 97/48814 herein incorporated by reference in their entirety).Those of skill in the art will appreciate the many advantages of the methods and compositions provided by the present invention. The following examples are included to demonstrate the preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### EXAMPLES

### EXAMPLE 1

Bacterial Strains and Plasmids

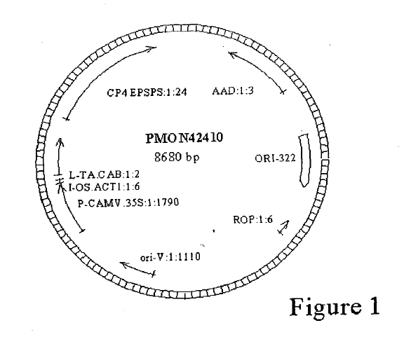

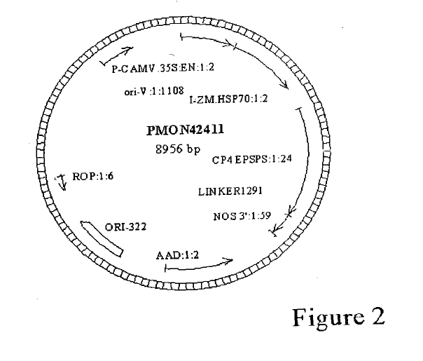

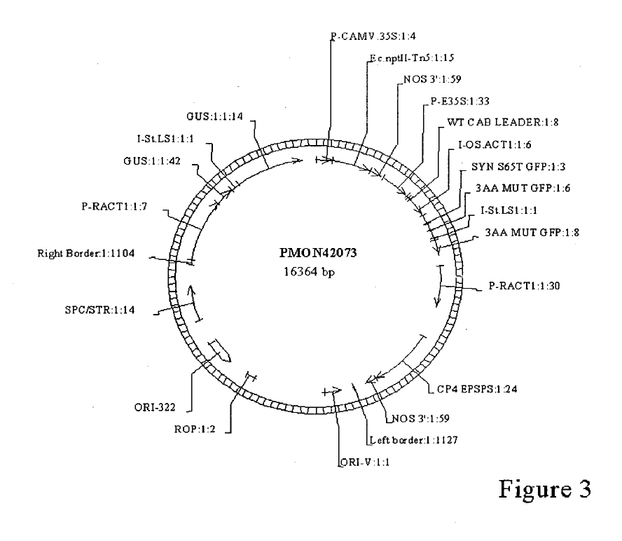

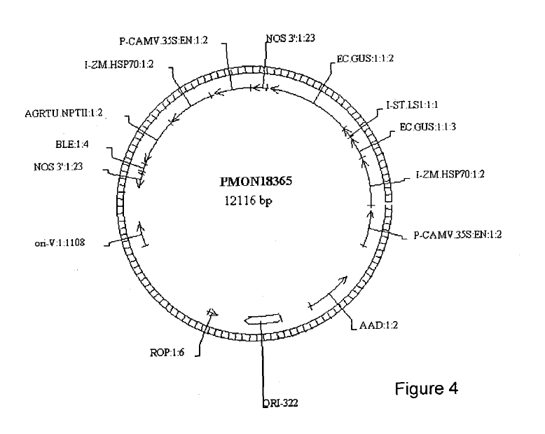

Agrobacterium tumefaciens strain ABI is harbored with binary vectors pMON42410 (FIG. 1) or pMON42411 (FIG. 2) or pMON42073 (FIG. 3) or pMON18365 (FIG. 4). The T-DNA of the vectors contain a neomycin phosphotransferase II gene (nptII) or CP4 EPSPS (glyphosate) as the selectable marker and a green fluorescence protein gene (gfp) or GUS as the screenable marker, driven by 35S promoter or the rice actin 1 promoter (P-RACT1) or the FMV promoter.

### EXAMPLE 2

Preparation of Agrobacterium for Corn Protocols

Agrobacterium ABI in glycerol stock is streaked out on solid LB medium supplemented with the antibiotics kanamycin (100 mg/L), spectinomycin (100 mg/L), streptomycin (100 mg/L) and chloramphenicol (25 mg/L) and incubated at 28° C. for 2 days. Two days before Agrobacterium inoculation, one loop of Agrobacterium cells from the LB plate is picked up and inoculated into 50 mL of liquid LB medium supplemented with 100 mg/L each of spectinomycin and kanamycin in a 250-mL flask. The flask is placed on a shaker at approximately 150 rpm and 27° C. overnight. The Agrobacterium culture is then diluted (1 to 5) in the same liquid medium and put back to the shaker. Several hours later in the late afternoon one day before inoculation, the Agrobacterium cells are spun down at 3500 rpm for 15 min. The bacterium cell pellet is re-suspended in induction broth with 200 μM of acetosyringone and 50 mg/L spectinomycin and 50 mg/L kanamycin and the cell density is adjusted to 0.2 at O.D. ₆₆₀. The bacterium cell culture (50 mL in each 250-mL flask) is then put back to the shaker and grown overnight. In the morning of inoculation day, the bacterium cells are spun down and washed with liquid ½ MS VI medium (Table 1) supplemented with 200 μM of acetosyringone. After one more spinning, the bacterium cell pellet is re-suspended in ½ MS PL medium (Table 1) with 200 μM of acetosyringone (Table 1), and the cell density is adjusted to 1.0 at O.D ₆₆₀ for inoculation. After resuspension, the Agrobacterium in ½ MSPL plus 200 μM of acetosyringone can be stored at 4° C. for up to 27 days and used as desired.

Reagents are commercially available and can be purchased from a number of suppliers (see, for example Sigma Chemical Co., St. Louis, Mo.).

TABLE 1
Media

| | | | | | Co-culture | | Induction | | | | | |
|---|---|---|---|---|---|---|---|---|---|---|---|---|
| Component | ½ MS VI | | ½ MS PL | | medium | | MS | | MS/BAP | | MSOD | |
| MS salts | 2.2 | g/l | 2.2 | g/l | 2.2 | g/l | 4.4 | g/l | 4.4 | g/l | 4.4 | g/l |
| Sucrose | 20 | g/l | 68.5 | g/l | 20 | g/l | 30 | g/l | 30 | g/l | — | |
| Maltose | — | | — | | — | | — | | — | | 40 | g/l |
| Glucose | 10 | g/l | 36 | g/l | 10 | g/l | — | | — | | 20 | g/l |
| 1-Proline | 0.115 | g/l | 0.115 | g/l | 0.115 | g/l | 1.36 | g/l | 136 | g/l | — | |
| Casamino Acids | — | | — | | — | | 0.05 | g/l | 0.05 | g/l | — | |
| Glycine | 2 | mg/l | 2 | mg/l | 2 | mg/l | — | | — | | — | |
| 1-Asparagine | — | | — | | — | | — | | — | | 150 | mg/l |
| myo-Inositol | 100 | mg/l | 100 | mg/l | 100 | mg/l | — | | — | | 100 | mg/l |
| Nicotinic Acid | 0.5 | mg/l | 0.5 | mg/l | 0.5 | mg/l | 0.65 | mg/l | 0.65 | mg/l | 0.65 | mg/l |
| Pyridoxine.HCl | 0.5 | mg/l | 0.5 | mg/l | 0.5 | mg/l | 0.125 | mg/l | 0.125 | mg/l | 0.125 | mg/l |
| Thiamine.HCl | 0.1 | mg/l | 0.1 | mg/l | 0.6 | mg/l | 0.125 | mg/l | 0.125 | mg/l | 0.125 | mg/l |
| Ca Pantothenate | — | | — | | — | | 0.125 | mg/l | 0.125 | mg/l | 0.125 | mg/l |
| 2,4-D | — | | — | | 3 | mg/l | 0.5 | mg/l | 0.5 | mg/l | — | |
| Picloram | — | | — | | — | | 2.2 | mg/l | 2.2 | mg/l | | |
| Silver Nitrate | — | | — | | — | | 3.4 | mg/l | — | | — | |
| Na-Thiosulfate | — | | — | | — | | — | | — | | — | |
| Phytagar | — | | — | | — | | 7.0 | g/l | 7.0 | g/l | 7.0 | g/l |
| Low EEO agarose | — | | — | | 5.5 | g/l | — | | — | | — | |

### EXAMPLE 3

Transformation of type I callus of an inbred corn line

Inoculation and co-culture: Immature embryos (1.0-2.0 mm) are isolated from sterilized ears and dipped into Agrobacterium cell suspension in 1.5-mL microcentrifuge tubes continuously for 15 minutes. The tube is then set aside for 5 min. After the Agrobacterium suspension is removed using a transfer pipette with fine tip, the embryos are transferred to standard co-culture medium (Table 1). The embryos are placed with the scutellum side facing up. The embryos are cultured in a Percival incubator set at 23° C. and dark for approximately 24 h.

Selection and regeneration and growth: After the co-cultivation, the embryos are transferred from the co-culture plates onto callus induction medium, induction MS (Table 1) with 500 mg/L carbenicillin and 100 or 200 mg/L paromomycin. The plates are kept in a dark culture room at 27° C. for approximately 2 weeks. Two weeks later, almost all the callus pieces developed individually are transferred onto MS6BAP (Table 1) with 250 mg/L carbenicillin and 100 or 200 mg/L paromomycin. The plates are kept in a culture room with 16-h light and at 27° C. for 5-7 days. Then, the callus pieces are transferred onto MSOD (Table 1) with 250 mg/L carbenicillin and 100 or 200 mg/L paromomycin. In another 2 weeks, all the pieces with shoots or living tissue are transferred onto the same media in phytatrays for further growth.

When the plantlets reach the lid and have a few roots, they are moved to soil in peat pots in a growth chamber. In 7 to 10 days, they are transplanted into 12-in pots and moved to the greenhouse with conditions for normal corn plant growth.

### EXAMPLE 4

Transformation of Type II Callus of an Inbred Corn Line

Inoculation and co-culture. Immature embryos are isolated from sterilized ears and directly dipped into the prepared Agrobacterium cell suspension in 1.5-mL microcentrifuge tube continuously for 15 min. The tube is then set aside for 5 min, which makes the inoculation time for individual embryos from 5 to 20 min. After Agrobacterium cell suspension is removed using a fine tipped sterile transfer pipette, the immature embryos are transferred onto the co-culture medium (Table 1). The embryos are placed on the medium with the scutellum side facing up. The embryos are cultured in a dark incubator (23° C.) for approximately 24 h.

Selection and regeneration: After the co-cultivation, the embryos are transferred onto callus induction medium, MS4C-2+SN (4.4 g/L MS basal salts, 10 mL/L MS vitamins 100×, 2.0 ml/L 2,4-D (1 mg/mL), 30 g/L sucrose, 2.72 g/LI-proline, 0.5 g/L MES, 2.5 g/L Phytagel, 1.7 mL/L silver nitrate (2 mg/mL)) supplemented with 0.5 mM glyphosate and 500 mg/L carbenicillin (MS4C-2+SN/gly0.5/C500). The culture plates are kept in a dark room at 27° C. for approximately 2 weeks. All callus pieces are transferred onto the fresh medium (the same medium) under the same conditions for another 2 weeks. All the callus pieces that are still growing or alive are transferred onto MS6BA medium (Table 1) supplemented with 0.25 mM glyphosate and 250 mg/L carbenicillin and incubated with 16-h light and at 27° C. for 5-7 days. The callus pieces are transferred onto MSOD media (Table 1) with 0.25 mM glyphosate and 250 mg/L carbenicillin in Petri dishes. In 2 weeks, all the pieces with shoots or living tissue are transferred onto MSOD with 0.25 mM glyphosate and 250 mg/L carbenicillin in phytatrays. When the plantlets reach the lid and have a few roots, they are moved to soil in peat pots in a growth chamber. In 7 to 10 days, they are transplanted into 12-in pots and moved to the greenhouse with conditions for normal corn plant growth.

### EXAMPLE 5

Transformation of Type II Callus of an Inbred Corn Line with Cold-stored Agrobacterium

A type II callus from a corn inbred line is transformed with pMON42410 and pMON42411 as described in Example 4 to evaluate the cold-stored Agrobacterium. The embryos are divided equally from each ear for both constructs to account for ear-to-ear variation. For example, if 4 ears are used, 50 embryos from each ear are used with each construct. Agrobacterium used is stored at 4° C. for 0, 1, 4, 7, and 14 days. The data suggest that transformation efficiencies similar to the standard 0 day control can be obtained by using Agrobacterium that has been stored up to 7 days. Table 2 summarizes transformation data from these experiments.

TABLE 2
Cold-stored Agrobacterium experiments.

| | | Transformants | |
|---|---|---|---|
| # Days | | containing | |
| Agrobacterium | Total # | pMON42410 or | % Transformation |
| stored at 4° C. | explants | pMON42411 | Efficiency |
| 0 | 398 | 24 | 6.03% |
| 1 | 403 | 10 | 2.48% |
| 4 | 198 | 19 | 9.59% |
| 7 | 207 | 22 | 1062% |
| 14 | 205 | 1 | 0.48% |

Table 3 shows results from transformation of a type II callus corn inbred line with pMON42073 based on GFP positive callus events.

TABLE 3
Efficacy of stared Agrobacterium pMON42073 based on
GFP positive plant events.

| # Days Stored | # Explants | # Plant Events | TE (%) |
|---|---|---|---|
| 0 | 140 | 17 | 12% |
| 1 | 121 | 11 | 9% |
| 4 | 135 | 14 | 10% |
| 7 | 134 | 52 | 39% |

Ninety-six of these events were regenerated. One plant per event was tested for GUS expression by leaf punch and GFP expression by leaf punch or root tip. Results are shown in Table 4.

TABLE 4
Anal sis of transgenic plants based on reporter gene expression.

| | |
|---|---|
| GFP Results | GUS Results |

| | Events | Events | Events | Events |
|---|---|---|---|---|
| Storage Time | Positive | Negative | Positive | Negative |
| Fresh | 17 | 0 | 16 | 1 |
| 1 Day | 11 | 0 | 8 | 3 |
| 4 Day | 14 | 1 | 14 | 1 |
| 7 Day | 52 | 1 | 48 | 5 |
| Total | 94 | 2 | 86 | 20 |

The experiment is repeated as before using Agrobacterium containing pMON42073 with storage times of 0 days (control), 5 days, 8 days, and 11 days at 4° C. Three Agrobacterium preparations are done for each storage time in order to reduce the effect that a specific Agrobacterium preparation would have on a given storage time. Results are presented in Table 5.

TABLE 5
Transformation with pMON42073 (CP4 selection, GFP activity and GUS
gene expression)

| | Explants | | | | |
|---|---|---|---|---|---|
| | # of | # of calli | | | TE (GFP+/Explants) |
| | embryos | survived | GFP+ | GUS+ | % |
| 0 days | 377 | 34 | 5 | 2 | 1.3 |
| 5 days | 369 | 20 | 5 | 2 | 1.4 |
| 8 days | 393 | 25 | 8 | 8 | 2.0 |
| 11 days | 383 | 36 | 9 | 7 | 2.3 |

Table 5 shows that TE (%) was calculated by number of GFP positive calli divide by total explants. Number of survived calli are not used for calculation of TE to aviod any problem of escape. It is clear that cold-stored Agrobacterium cells can improve transformation efficiency.

Cold and stored Agrobacterium (up to 11 days at 4° C.) can be used for corn transformation without sacrificing the TE. Instead, after storage at 4° C. for 7-11 day, the Agrobacterium cell can improve transformation efficiency up to 43% (Table 5). In some other experiments, the TE with stored Agrobacterium was even 3-fold higher than that with freshly isolated Agrobacterium cells.

There is no significant difference in terms of insert copy number and insert quality between fresh and 7-day stored Agrobacterium according to a Southern blot analysis (Southern, J. Mol. Biol., 98:503-517, 1975).

Type I callus from a corn inbred line was transformed as described in Example 3 with pMON18365 (FIG. 4). Table 6 shows that Agrobacterium cold storage also works in another corn transformation system.

TABLE 6
Transformation with pMON18365 (nptII selection and GUS gene
expression)

| # Days Stored | # Explants | # Plant Events | TE (%) |
|---|---|---|---|
| 0 | 70 | 1 | 1.4 |
| 2 | 70 | 3 | 4.2 |
| 4 | 70 | 4 | 5.7 |
| 8 | 70 | 3 | 4.2 |

### EXAMPLE 6

Testing Agrobacterium Viability in Various Media

Agrobacterium can be tested for its ability to be stored in various media by varying the osmotic pressure of the media and looking for bacterial viability. The control here is the same ½ MS medium as others but no sugars included. In the case of corn, this is ½ MS media plus MS vitamins and proline with 200 μM acetosyringone. Then Agrobacterium in ½ MS PL media containing (1) no sugars (CK), (2) 1% glucose, 2% sucrose and (3) 3% glucose, 3% sucrose and (4) 3.6% glucose, 6.8% sucrose and (5) 3.6% glucose, 10% sucrose and (6) 3.6% glucose, and 15% sucrose and (7) 3.6% glucose, and 20% glucose is stored for 0, 1 day, 7 days, 14 days, and 21 days at 4° C. Viability is tested by culturing Agrobacterium cells on a LB solid medium (plates) and then counting its colonies per mL (culture on the plate) in each treatment. Table 7 shows that osmotic pressure has little effect on bacterium survivability.

TABLE 7
Bacterium survivability after storage in cold environment (4° C.) for
0, 1, 7, 14 or 21 days. (½ MS medium plus MS vitamins and praline
as well as acetosyringone (200 μM))

| | % | % | | | | | | |
|---|---|---|---|---|---|---|---|---|
| Treat- | Glu- | Su- | | # 0 | colonies | (×10⁸) | 14 | 21 |
| ment | cose | crose | | days | 1 day | 7 days | days | days |
| 1 | 0 | 0 | CK | 11.8 | 8.5 | 6 | 4.6 | 0.9 |
| 2 | 1 | 2 | ½ | 8.1 | 6.9 | 3.5 | 0.5 | 0.1 |
| | | | MSVI | | | | | |
| 3 | 3 | 3 | | 5.8 | 6.4 | 4.7 | 1.7 | 0.1 |
| 4 | 3.6 | 6.85 | ½ | 3.7 | 4 | 4.7 | 3.1 | 0.3 |
| | | | MSPL | | | | | |
| 5 | 3.6 | 10 | | 9.1 | 7.2 | 4.3 | 4.8 | 0.4 |
| 6 | 3.6 | 15 | | 5.6 | 4.4 | 5.4 | 4.2 | 0.3 |
| 7 | 3.6 | 20 | | 7.4 | 4.9 | 4.4 | 3 | 0.3 |

1. A method of preparing an Agrobacterium inoculation culture for use in transformation of monocotyledonous plant cells or plant tissue comprising the steps of:
providing an Agrobacterium inoculation culture at a plant tissue inoculation density;
storing the Agrobacterium inoculation culture in a cold environment for at least about 12 hours to obtain a chilled Agrobacterium inoculation culture; and
combining the chilled Agrobacterium inoculation culture with plant cells or plant tissue in a manner suitable to achieve transformation of said plant cells or plant tissue.

2. The method of claim 1 additionally comprising producing a transformed plant from the inoculated plant cells or plant tissue.

3. The method of claim 1 in which the cold environment is from about 0° C. to about 12° C.

4. The method of claim 1 in which the cold environment is from about 1° C. to about 6° C.

5. The method of claim 1 in which the cold environment is about 4° C.

6. The method of claim 1 in which the chilled Agrobacterium inoculation culture is stored from about 12 hours to about 30 days.

7. The method of claim 1 in which the chilled Agrobacterium inoculation culture is stored from about 3 days to about 14 days.

8. The method of claim 1 in which the chilled Agrobacterium inoculation culture is stored from about 4 days to about 7 days.

9. A transformed plant produced from the method of claim 1.

10. A method of preparing an Agrobacterium inoculation culture for use in the transformation of monocotyledonous plant cells or plant tissue comprising the steps of:
providing Agrobacterium inoculation culture at a plant tissue inoculation density;
storing the Agrobacterium inoculation culture at a temperature of about 4° C. for a time period from about 4 days to about 7 days to obtain a chilled Agrobacterium inoculation culture; and
combining the chilled Agrobacterium inoculation culture with plant cells or plant tissue in a manner suitable to achieve transformation of said plant cells or plant tissue.
